(19) United States
(12) Patent Application Publication
(10) Pub. No.: US 2012/0316539 A1
(43) Pub. Date: Dec. 13, 2012

(54) Catheter Clamp

(76) Inventor: Cuauhtemoc Villasana, Dallas, TX (US)

(21) Appl. No.: 13/158,455

(22) Filed: Jun. 13, 2011

(51) Int. Cl. A61M 27/00

(52) U.S. Cl. 604/544

(57) ABSTRACT

A catheter clamp having a rounded open proximal side, a mirroring distal end, a planar bottom surface, and a top surface is provided. The top surface extends from the proximal side toward the distal end, the proximal side of the catheter clamp being formed such that the top surface can be moved in a vertically swinging position. The distal end of the clamp includes a locking lip that extends from the upper inside surface of the distal end. The top surface has a clamping surface which extends in a perpendicular direction from the top surface. The top surface is engaged beneath the locking lip such that the clamping surface clamps an inserted catheter tube, thereby stopping the flow of liquids through the catheter clamp. The top surface can be easily disengaged to release liquids through the catheter tube.

### BACKGROUND

1. Field of the Invention

The present invention relates generally to methods of clamping catheters and in particular to a clamp suitable for use with urinary catheters, which are most often inserted into a person's bladder via the urethra, the clamp being placed on the portion of the catheter extending outside the body and which allows the catheter to be easily clamped to prevent flow through the catheter, and easily released when a person is ready to discharge urine through the catheter, thus enabling the person to live a more normal life without being dependent on drainage bags.

Within the medical industry, and in particular within the catheter market, urinary catheterization may be required. Urinary catheters are used to drain the bladder and may be required when a person has urinary incontinence, urinary retention, when surgery makes a urinary catheter necessary, such as prostate or gynecological surgery, and often as a result of other medical conditions, such as multiple sclerosis, spinal cord injury, or dementia.

A urinary catheter is a tube system that is placed in the body to drain and collect urine from the bladder. In urinary catheterization, a plastic tube known as a urinary catheter is most often slid into a person's bladder via the urethra. Catheterization allows a person's urine to drain freely from the bladder for collection. The catheterization procedure is usually done by a clinician, such as a nurse, but self-catheterization is common as well.

Catheters come in a variety of sizes, materials, and types.

Catheter diameters are sized by the French catheter scale (F). The most common sizes range from 10 F (3.3 mm) to 28 F (9.3 mm). The clinician selects a size large enough to allow free flow of urine, and large enough to control leakage of urine around the catheter. A larger size can become necessary when the urine is thick, bloody or contains large amounts of sediment. Larger catheters, however, are more likely to cause damage to the urethra.

The most common material for a catheter is latex. However, some people develop allergies or sensitivities to latex after long-term latex catheter use making it necessary to use catheters made from another material, such as silicone or coated with Teflon.

There are two main types of tube catheters: indwelling and intermittent.

An indwelling urinary catheter is typically left in place in the bladder and is most often used for an extended period of time. Indwelling catheters attach to a drainage bag to collect the urine. An indwelling catheter is most often inserted through the urethra, which is the tube that brings urine from the bladder to the outside of the body. However, sometimes a tube, called a suprapubic catheter, is inserted into a person's bladder from a small hole in the person's belly. An indwelling catheter has a small balloon inflated on the end of it. This prevents the catheter from sliding out of the body. When it's necessary to remove the catheter, the balloon is deflated.

Intermittent catheters are typically used in accordance with a specific periodic time frame where the intermittent catheter is inserted to empty the bladder and removed after the flow of urine has stopped.

Catheters are usually attached to a drainage bag. A leg bag is a smaller drainage bag that attaches, usually by elastic band, to the leg. It is usually worn during the day because it fits discretely under the pants or skirts. When the leg drainage bag is full, it is emptied into a toilet. A down drain is a larger drainage device that is usually used during the night. The down drain bag is typically hung on a hook installed on the bottom of the bed. These drainage bags must always stay lower than the bladder to prevent urine from flowing back into the bladder, which can cause a variety of infections.

The duration of catheterization can have a significant impact on the patient. Long-term catheterization carries a significant risk of urinary tract infection. Other long term complications may include blood infections, known as sepsis, urethral injury, skin breakdown, bladder stones, and blood in the urine, known as hematuria. In addition, a person may develop an allergy or sensitivity to latex, blood infections, or kidney damage. Finally, the tube or a urinary catheter can become clogged or kinked, which may lead to several of the above complications.

To combat infection, every day care is required. For example, a person that is catheterized should clean the urethral area where the catheter exits the body and the catheter itself, disconnect the drainage bag only with clean hands and as seldom as possible, keep the drainage bag connector as clean as possible, and drink sufficient liquid to fill and empty the bladder several times each day.

Using an intermittent catheter can be inconvenient because of the need to catheterize, empty the bladder, and clean up to six times a day. In addition, fluid intake must be monitored and limited to certain times of the day to regulate the amount of urine that is in the bladder. However, there are significant advantages. Overfilling of the bladder decreases circulation to the bladder walls, which increases the risk of reflux of colonized urine to the kidneys, a cause of urinary tract infections. Bacteria that commonly cause urinary tract infections can double in number in as little as forty-five minutes to an hour. This is a geometric progression and quickly increases the number of bacteria in the urine. Therefore, catheterizing more often keeps the bacterial concentration lower. Additionally, the use of an intermittent catheter is a more “natural” way of filling and emptying of the bladder and results in less bladder shrinkage. There is also no need to wear drainage bags.

If intermittent catheterizing is done correctly, it also has the least risk of damage to the upper urinary tract, lowest risk of serious urinary tract infections, lowest risk of stones, and lowest risk of bladder cancer, all due to the fact that the bladder is emptied on a regular schedule and urine does not stay in the bladder. Indwelling catheters significantly increase the risk for all of these complications and also reduce fertility in males.

2. Description of Related Art

Catheter clamps are generally used to clamp the catheter tubing to stop the flow of fluids through the catheter. A variety of catheter clamps exist for various purposes. One such catheter clamp is used to clamp an inserted indwelling catheter while the drainage bag is changed. Another variety of existing catheter clamps is used to secure a catheter tube to a certain part of the body so that the catheter does not move in or out. The existing catheter clamps address only the need to fix the catheter at a certain location or to stop the flow of liquid through the catheter while maintenance is performed. None of the existing catheter clamps address the advantages of both intermittent catheter use and indwelling catheter use.

A need exists, therefore, for a catheter clamp that will allow the convenience of the indwelling catheter while addressing the advantages of the intermittent catheter.

All references cited herein are incorporated by reference to the maximum extent allowable by law. To the extent a reference may not be fully incorporated herein, it is incorporated by reference for background purposes and indicative of the knowledge of one of ordinary skill in the art.

### BRIEF SUMMARY OF THE INVENTION

The problems presented in existing indwelling and intermittent catheter use and the clamps used therefore are solved by the systems and methods of the present invention. In accordance with one embodiment of the present invention, a catheter clamp is provided. The catheter clamp has a proximal side with a rounded opening, through which a catheter tube can be inserted. The catheter clamp has a mirroring distal end with a rounded opening which allows a catheter tube to be inserted through the proximal side and protrude through the distal end. The openings of the proximal side and distal end are of a size that allows the clamp to stay fitted on the catheter tube whether the catheter clamp is in the open or closed position. The bottom surface of the catheter clamp has a planar surface which supports the catheter tubing. The top surface of the catheter clamp extends from the top of the proximal side toward the distal end, the proximal side of the catheter clamp being formed such that the top surface can be moved in a vertically swinging position to allow the catheter tubing to be inserted when the catheter clamp is open, or in the disengaged position. The distal end of the clamp has a locking lip that extends from the upper inside surface of the distal end. The top surface has a clamping surface which extends in a perpendicular direction from the top surface. When the top surface is pressed down, the top surface engages beneath the locking lip of the distal end of the catheter clamp. When in this closed, or engaged, position the clamping surface presses down on the catheter tubing against the planar bottom surface such that the flow of liquids through the catheter clamp is stopped. To release liquids through the catheter tube, the top surface is disengaged from the locking lip of the distal end of the catheter clamp.

The use of this new and improved catheter clamp allows a person who has an indwelling catheter to periodically empty the bladder without the need for a drainage bag. The catheter clamp is simply disengaged and the bladder is emptied through the catheter tube into a toilet and then engaged to again stop the flow of liquids. Therefore, a catheterized person enjoys the convenience advantages of an indwelling catheter without the stigma of wearing a drainage bag or being connected to a down drain, allowing the person to empty the bladder on a regular basis, which provides the health advantages of an intermittent catheter, all without the need to monitor fluid intake.

Other objects, features, and advantages of the present invention will become apparent with reference to the drawings and detailed description that follow.

### BRIEF DESCRIPTION OF THE DRAWINGS

FIG. 1 is a pictorial representation of a top perspective view of the catheter clamp in the open, or disengaged, position in accordance with an illustrative embodiment.

FIG. 2 is a pictorial representation of a left side view of the catheter clamp in the open, or disengaged, position in accordance with an illustrative embodiment.

FIG. 3 is a pictorial representation of a top perspective view of the catheter clamp in the closed, or engaged, position in accordance with an illustrative embodiment.

FIG. 4 is a pictorial representation of a left side view of the catheter clamp in the closed, or engaged, position in accordance with an illustrative embodiment.

### DETAILED DESCRIPTION OF THE PREFERRED EMBODIMENT

All references cited herein are incorporated by reference to the maximum extent allowable by law. To the extent a reference may not be fully incorporated herein, it is incorporated by reference for background purposes and indicative of the knowledge of one of ordinary skill in the art.

In the following detailed description of the preferred embodiments, reference is made to the accompanying drawings which form a part hereof, and in which is shown by way of illustration specific preferred embodiments in which the invention may be practiced. These embodiments are described in sufficient detail to enable those skilled in the art to practice the invention, and it is understood that other embodiments may be utilized and that logical mechanical changes may be made without departing from the spirit or scope of the invention. To avoid detail not necessary to enable those skilled in the art to practice the invention, the description may omit certain information known to those skilled in the art. The following detailed description is, therefore, not to be taken in a limiting sense, and the scope of the present invention is defined only by the appended claims.

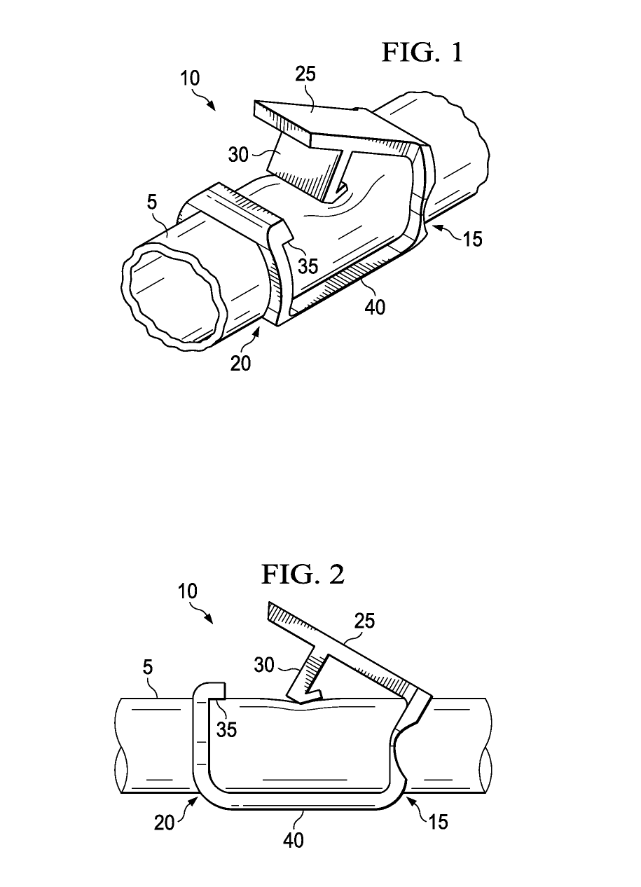

With reference now to the figures, and in particular, with reference to FIGS. 1-4, exemplary diagrams of environments are provided in which illustrative embodiments may be implemented. It should be appreciated that FIGS. 1-4 are only exemplary and are not intended to assert or imply any limitation with regard to the environments in which different embodiments may be implemented. Many modifications to the depicted environments may be made.

FIG. 1 is a pictorial representation of a top perspective view of the catheter clamp in the open, or disengaged, position in accordance with an illustrative embodiment. The catheter clamp 10 is fitted on a catheter tube 5. The catheter clamp 10 can be made of any sturdy, lightweight material such as polyvinyl chloride. The catheter clamp 10 is approximately 1½ inches in length. The catheter clamp 10 has a proximal side 15 with a rounded opening. The rounded opening of the proximal side 15 is approximately ¾ inches in diameter. The proximal side 15 has rounded sides and a planar top and bottom. This configuration allows the catheter clamp 10 to be firmly fitted onto the catheter tube 5. The catheter clamp 10 has a mirroring distal end 20 with rounded sides and a planar top and bottom. The rounded sides of the proximal side 15 and the distal end 20 provide comfort to the user. The catheter clamp 10 can be fitted on a catheter tube 5 by inserting the catheter tube 5 through the proximal side 15 until the catheter tube 5 protrudes through the distal end 20. The catheter tube 5 will protrude through the distal end 20 of the catheter clamp 10 a desired length, approximately ⅙ of an inch, such that the user can easily direct the flow of liquid through the catheter tube 5. The ability of the catheter tube 5 to protrude a certain distance through the catheter clamp 10 is also suggested to minimize damage to the catheter tube 5. The openings of the proximal side 15 and distal end 20 are of a size that allows the catheter clamp 10 to stay fitted on the catheter tube 5 whether the catheter clamp 10 is the open or closed position. One skilled in the art will understand that the materials and dimensions provided in the illustrative embodiment are exemplary and may be adjusted without taking away the inventive properties of the present invention.

The bottom surface 40 of the catheter clamp 10 has a planar surface which supports the catheter tubing 5. The top surface 25 of the catheter clamp 10 extends from the top of the proximal side 15 toward the distal end 20, the proximal side 15 of the catheter clamp 10 being formed such that the top surface 25 can be moved in a vertically swinging position to allow the catheter tubing 5 to be inserted when the catheter clamp 10 is open, or in the disengaged position. The distal end 20 of the catheter clamp 10 has a locking lip 35 that extends from the upper inside surface of the distal end 20. The top surface 25 has a clamping surface 30 which extends in a perpendicular direction from the top surface 25. When the top surface 25 is pressed down, the top surface 25 engages beneath the locking lip 35 of the distal end 20 of the catheter clamp 10. When in this closed, or engaged, position the clamping surface 30 presses down on the catheter tubing 5 against the planar bottom surface 40 such that the flow of liquids through the catheter tube 5 is stopped. To release liquids through the catheter tube 5, the top surface 25 is disengaged from the locking lip 35 of the distal end 20 of the catheter clamp 10.

FIG. 2 is a pictorial representation of a left side view of the catheter clamp in the open, or disengaged, position in accordance with an illustrative embodiment. The catheter tube 5 extends through the proximal side 15 of the catheter clamp 10, through the distal end 20 of the catheter clamp 10, and protrudes approximately ⅙ inch in length. In this open, or disengaged, position liquid can flow through the catheter tube 5. In such an example, a user can empty the bladder through the catheter tube 5 into a toilet without having to drain into a bag or having to catheterize just to empty the bladder.

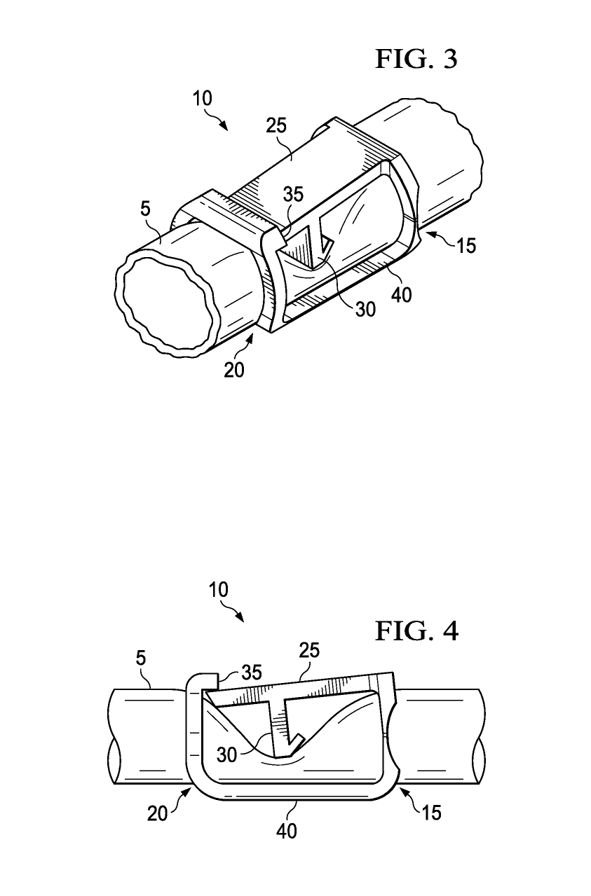

FIG. 3 is a pictorial representation of a top perspective view of the catheter clamp in the closed, or engaged, position in accordance with an illustrative embodiment. The catheter clamp 10 is fitted on a catheter tube 5. The catheter tube 5 protrudes through the distal end 20 of the catheter clamp 10 approximately ⅙ of an inch, such that the user can easily direct the flow of liquid through the catheter tube 5. The top surface 25 of the catheter clamp 10 extends from the top of the proximal side 15 toward the distal end 20, the proximal side 15 of the catheter clamp 10 being formed such that the top surface 25 can be moved in a vertically swinging position to allow the catheter tubing 5 to be inserted when the catheter clamp 10 is open, or in the disengaged position. The distal end 20 of the catheter clamp 10 has a locking lip 35 that extends from the upper inside surface of the distal end 20. The top surface 25 has a clamping surface 30 which extends in a perpendicular direction from the top surface 25. When the top surface 25 is pressed down as in this FIG. 3, the top surface 25 engages beneath the locking lip 35 of the distal end 20 of the catheter clamp 10. When in this closed, or engaged, position the clamping surface 30 presses down on the catheter tubing 5 against the planar bottom surface 40 such that the flow of liquids through the catheter tube 5 is stopped. To release liquids through the catheter tube 5, the top surface 25 is disengaged from the locking lip 35 of the distal end 20 of the catheter clamp 10.

FIG. 4 is a pictorial representation of a left side view of the catheter clamp in the closed, or engaged, position in accordance with an illustrative embodiment. The catheter tube 5 extends through the proximal side 15 of the catheter clamp 10, through the distal end 20 of the catheter clamp 10, and protrudes approximately ⅙ inch in length. In this closed, or engaged, position liquid cannot flow through the catheter tube 5. In such an example, a user can have the catheter clamp 10 engaged and resume normal daily activity without having to wear a drain bag or having to catheterize just to empty the bladder. When a user needs to empty the bladder, either through feeling the bladder full or at a specific periodic interval, the user simply goes to a bathroom, releases the top surface 25 from the locking lip 35, the top surface 25 swings upward vertically thereby releasing the clamping surface 30 from the catheter tube 5, and the user can allow the bladder to empty through the catheter tube 5 into a toilet.

It should be apparent from the foregoing that an invention having significant advantages has been provided. While the invention is shown in only a few of its forms, it is not just limited but is susceptible to various changes and modifications without departing from the spirit thereof.

I claim:

1. A catheter clamp comprising:
a proximal side with a rounded opening, through which a catheter tube can be inserted;
a mirroring distal end with a rounded opening which allows a catheter tube to be inserted through the proximal side and protrude through the distal end;
a bottom planar surface that supports the catheter tubing;
a top surface of the catheter clamp that extends from the top of the proximal side toward the distal end;
the proximal side of the catheter clamp being formed such that the top surface can be moved in a vertically swinging position to allow the catheter tubing to be inserted when the catheter clamp is in the disengaged position;
a locking lip that extends from the upper inside surface of the distal end; and
a clamping surface which extends in a perpendicular direction from the top surface.

2. The catheter clamp according to claim 1 wherein:
the openings of the proximal side and distal end being of a size that allows the clamp to stay fitted on the catheter tube whether the catheter clamp is the open or closed position.

3. The catheter clamp according claim 1 wherein:
the sides of the proximal side and distal end are smooth and rounded for the comfort of the user.

4. A catheter clamp comprising:
a proximal side with a rounded opening, through which a catheter tube can be inserted;
a mirroring distal end with a rounded opening which allows a catheter tube to be inserted through the proximal side and protrude through the distal end;
the openings of the proximal side and distal end being of a size that allows the clamp to stay fitted on the catheter tube whether the catheter clamp is the open or closed position;
the sides of the proximal side and distal end are smooth and rounded for the comfort of the user;
a bottom planar surface that supports the catheter tubing;
a top surface of the catheter clamp that extends from the top of the proximal side toward the distal end;
the proximal side of the catheter clamp being formed such that the top surface can be moved in a vertically swinging position to allow the catheter tubing to be inserted when the catheter clamp is in the disengaged position;
a locking lip that extends from the upper inside surface of the distal end;
a clamping surface which extends in a perpendicular direction from the top surface;
the top surface being able to be temporarily locked beneath the locking lip of the distal end of the catheter clamp such that the clamping surface compresses the catheter tubing against the bottom wall of the catheter tubing, supported by the planar bottom, such that the flow of liquids through the catheter clamp is stopped.

5. A method for using a catheter clamp with an indwelling catheter, the method comprising:
inserting a catheter tube through the proximal side of a catheter clamp having a rounded opening;
moving the catheter clamp along the catheter tube such that the catheter tube protrudes through the mirroring distal end of the catheter clamp;
the catheter clamp having openings of the proximal side and distal end being of a size that allows the clamp to stay fitted on the catheter tube whether the catheter clamp is the open or closed position;
the catheter clamp further having a bottom planar surface that supports the catheter tubing and a top surface that extends from the top of the proximal side toward the distal end, the proximal side of the catheter clamp being formed in such a way that allows the top surface to be moved in a vertically swinging position, a locking lip that extends from the upper inside surface of the distal end, and a clamping surface which extends in a perpendicular direction from the top surface;
engaging the top surface beneath the locking lip of the distal end of the catheter clamp, thereby compressing the catheter tubing against the bottom wall of the catheter tube, supported by the planar bottom, such that the flow of liquids through the catheter clamp is stopped;
at a predetermined periodic interval, disengaging the top surface from the locking lip of the distal end of the catheter clamp such that liquids can flow through the catheter tube;
emptying the bladder through the catheter tube; and
engaging the top surface beneath the locking lip of the distal end of the catheter clamp, thereby compressing the catheter tubing against the bottom wall of the catheter tube, supported by the planar bottom, such that the flow of liquids through the catheter clamp is stopped.
